(19) United States
(12) Patent Application Publication
(10) Pub. No.: US 2013/0123523 A1
(43) Pub. Date: May 16, 2013

(54) METHODS FOR THE PREPARATION OF ETONOGESTREL AND DESOGESTREL

(71) Applicants: Klaus Nickisch, Berlin (DE); Bindu Santhamma, San Antonio, TX (US)

(72) Inventors: Klaus Nickisch, Berlin (DE); Bindu Santhamma, San Antonio, TX (US)

(21) Appl. No.: 13/293,656

(22) Filed: Nov. 10, 2011

(51) Int. Cl. C07J 75/00; C07J 1/00

(52) U.S. Cl. 552/505; 552/526; 552/618

(57) ABSTRACT

Described herein is a process for the synthesis of etonogestrel and desogestrel and intermediates used to form etonogestrel and desogestrel.

### BACKGROUND OF THE INVENTION

1. Field of the Invention

The present invention generally relates to processes for the synthesis of desogestrel and etonogestrel.

2. Description of the Relevant Art

Desogestrel and etonogestrel are synthetic steroids with strong progestational activity. Desogestrel is currently used as a synthetic progestin in oral contraceptive formulations, whereas etonogestrel is being used as synthetic progestin in the vaginal ring delivery system NuvaRing® and in the implant Implanon®.

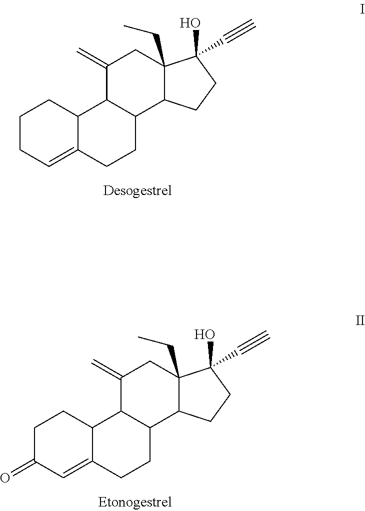 [SMILES: C#C[C@]1(O)CCC2C3CCC4=CCCCC4C3C(=C)C[C@@]21CC ; C#C[C@]1(O)CCC2C3CCC4=CC(=O)CCC4C3C(=C)C[C@@]21CC]

Numerous synthetic routes have been described in the literature for the synthesis of these compounds.

The synthesis of desogestrel was described for the first time in the German patent DE 2.361.120, which is incorporated herein by reference. This route is long and troublesome because the 13-ethyl moiety was introduced via oxidation of the 18-methyl group (Scheme 1).

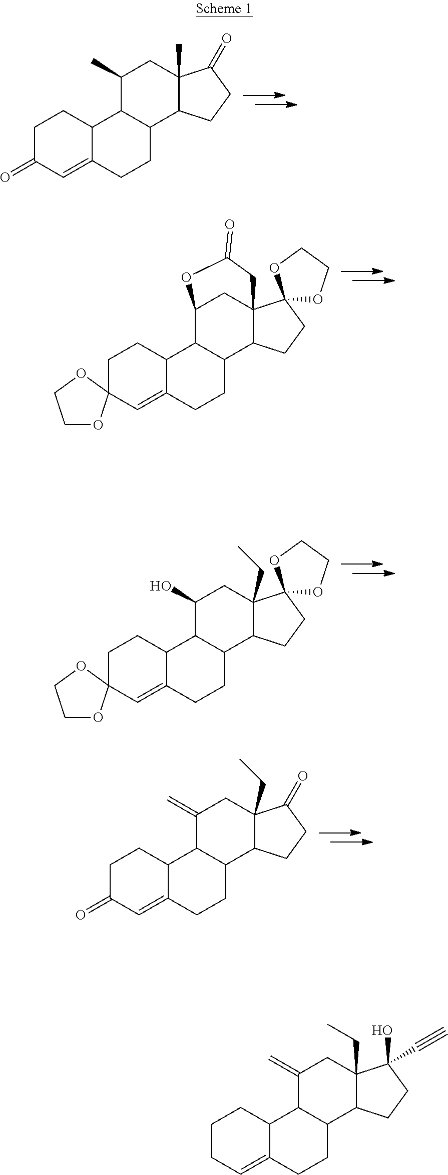

Recently, a new total synthesis of desogestrel was reported WO 2009/033499, which is incorporated herein by reference, where palladium catalyzed cyclization reaction was employed as the key step in the synthesis (Scheme 2).

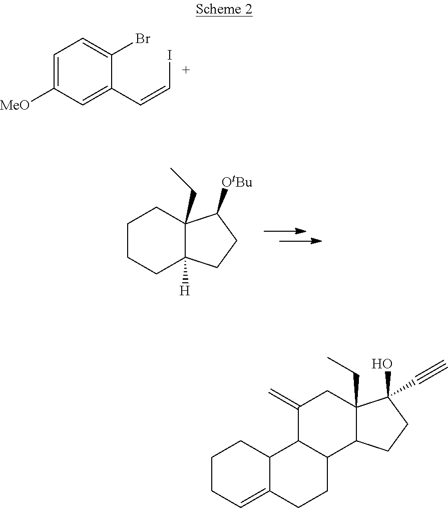

Both the methods are however not suitable for large scale production in view of their prolonged synthesis as well as complexity of the steps involved.

A more practical approach is to start the synthesis with intermediate III.

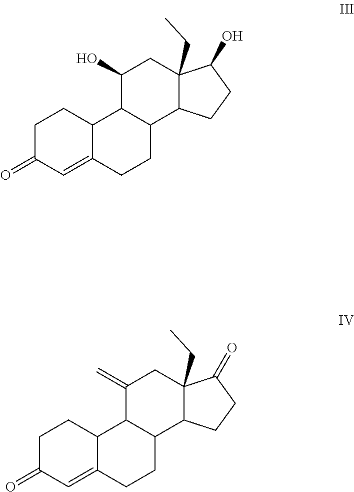 [SMILES: CC[C@]12C[C@H](O)C3C4CCC(=O)C=C4CCC3C1CC[C@@H]2O ; C=C1C[C@]2(CC)C(=O)CCC2C2CCC3=CC(=O)CCC3C12]

III could be conveniently synthesized by introducing the 11-hydroxy group through microbiological hydroxylation (EP 0144984, WO 2004/014934, both of which are incorporated herein by reference) which was then transformed to the key intermediate IV and finally to desogestrel and etonogestrel.

It should be however noted that, industrial scale microbiological transformations are troublesome since these reactions are normally performed in rather low concentrations and require the use of big and expensive equipments.

The goal of this invention was therefore to come up with a synthesis that would produce the key intermediate IV of desogestrel and etonogestrel in an efficient manner using easily available starting materials and applying reaction conditions suitable for large scale production without the need to apply microbiological transformations.

Such a concept has been already described in the literature (Steroids 62. 398-402, 1997 and Synthetic Communication 26 (23) 4437-4446 1996) (Scheme 3), both of which are incorporated herein by reference.

However, certain steps of this sequence did not work in our hands as claimed viz the elimination of 11-hydroxy group under formic acid reflux conditions led to mixtures of isomeric olefins delta 9,11, delta 12 and 11 methylene that were very hard to separate.

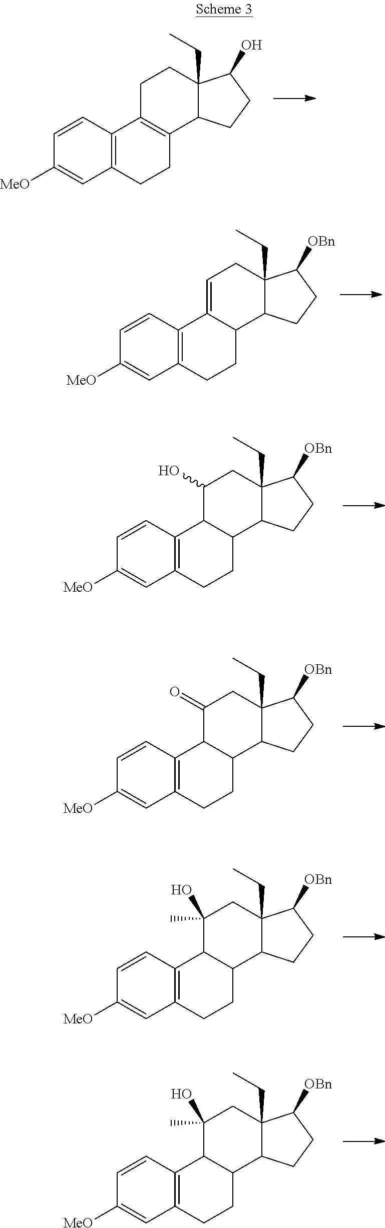 [SMILES: CC[C@]12CCC3=C(CCc4cc(OC)ccc43)C1CC[C@@H]2O ; CC[C@]12CC=C3c4ccc(OC)cc4CCC3C1CC[C@@H]2OCc1ccccc1 ; CC[C@]12CC(O)C3c4ccc(OC)cc4CCC3C1CC[C@@H]2OCc1ccccc1 ; CC[C@]12CC(=O)C3c4ccc(OC)cc4CCC3C1CC[C@@H]2OCc1ccccc1 ; CC[C@]12C[C@](C)(O)C3c4ccc(OC)cc4CCC3C1CC[C@@H]2OCc1ccccc1 ; CC[C@]12C[C@](C)(O)C3c4ccc(OC)cc4CCC3C1CC[C@@H]2OCc1ccccc1]

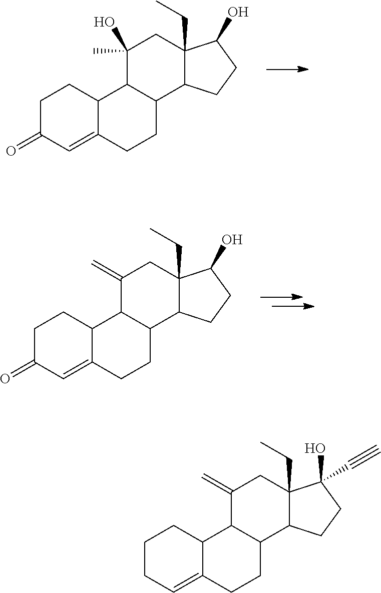 [SMILES: CC[C@]12C[C@](C)(O)C3C4CCC(=O)C=C4CCC3C1CC[C@@H]2O ; C=C1C[C@@]2(CC)C(CC[C@@H]2O)C2CCC3=CC(=O)CCC3C12 ; C#C[C@]1(O)CCC2C3CCC4=CCCCC4C3C(=C)C[C@@]21CC]

### SUMMARY OF THE INVENTION

Described herein is a method for the synthesis of etonogestrel and desogestrel that includes:
converting compound 1 to compound 6;

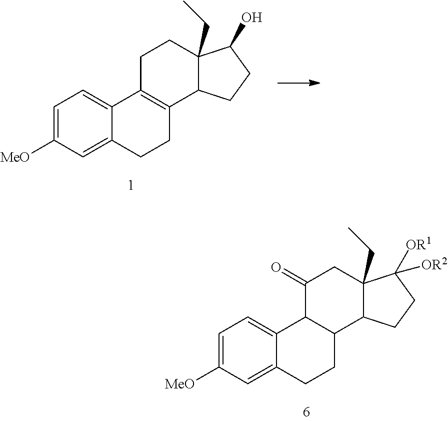

where R¹ and R² are independently C₁-C₆ straight chain or branched alkyl or R¹ and R² together combine to form a cyclic ketal protecting group forming a 5 or 6 members cyclic group comprising an unbranched or branched hydrocarbon;
converting compound 6 to compound 9;

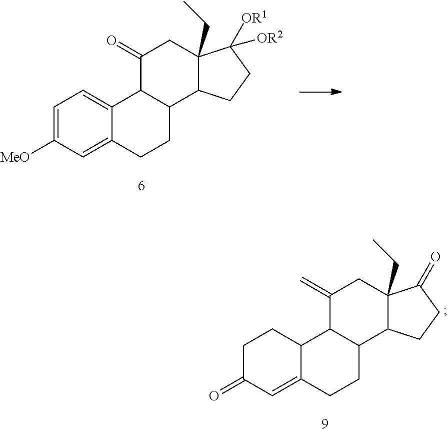

and
converting compound 9 into etonogestrel or desogestrel.

In one embodiment, converting compound 1 to compound 6 includes obtaining or synthesizing compound 1;
oxidizing compound 1 to form compound 2;

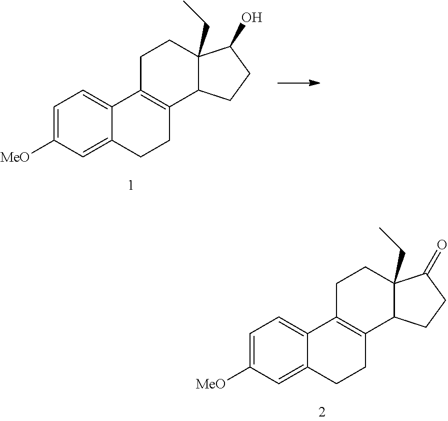 [SMILES: CC[C@]12CCC3=C(CCc4cc(OC)ccc43)C1CC[C@@H]2O ; CC[C@]12CCC3=C(CCc4cc(OC)ccc43)C1CCC2=O]

treating compound 2 with an acid to form the isomerized intermediate 3; and

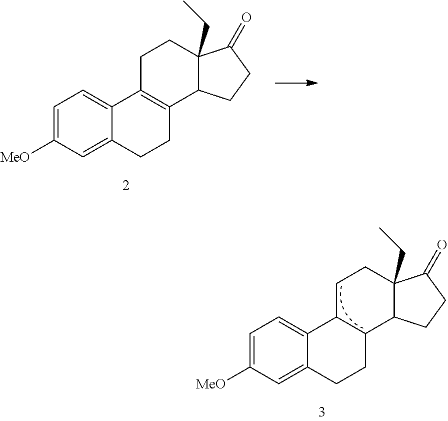 [SMILES: CC[C@]12CCC3=C(CCc4cc(OC)ccc43)C1CCC2=O ; CC[C@]12CCC3c4ccc(OC)cc4CCC3C1CCC2=O]

forming the ketal protected intermediate 4 from compound 3

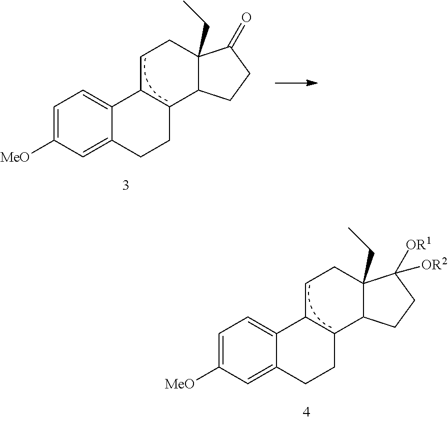

hydroxylating compound 4 to form hydroxy intermediate 5

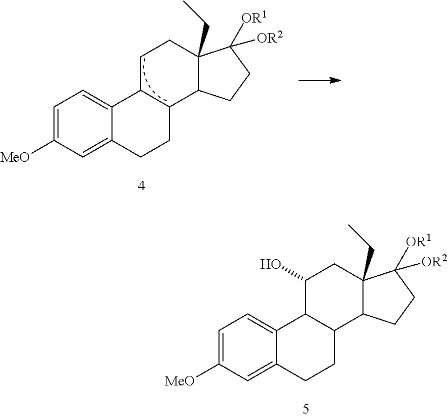

oxidizing the hydroxyl intermediate 5 to form compound 6

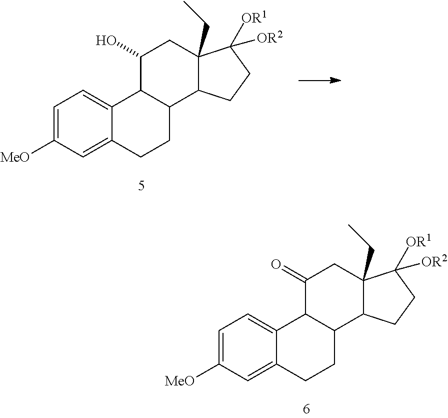

In an embodiment, converting compound 6 to compound 9 includes forming silyl intermediate 7 from compound 6

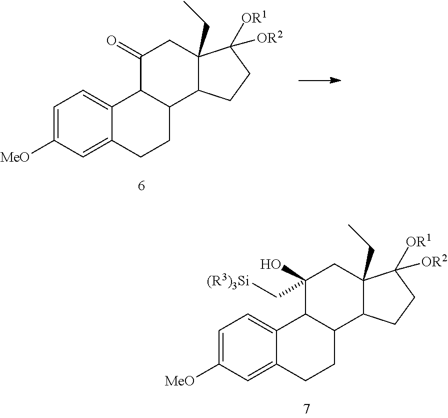

where R³ is an alkyl group.
reducing silyl intermediate 7 to form compound 8

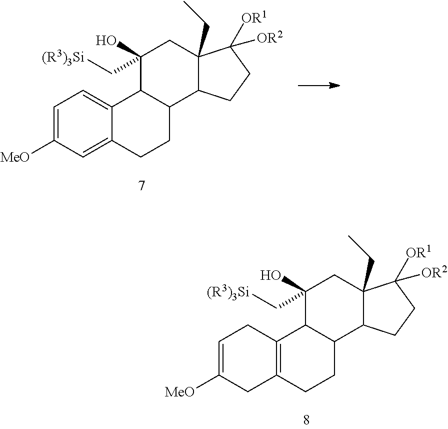

and
treating compound 8 with acid to form compound 9

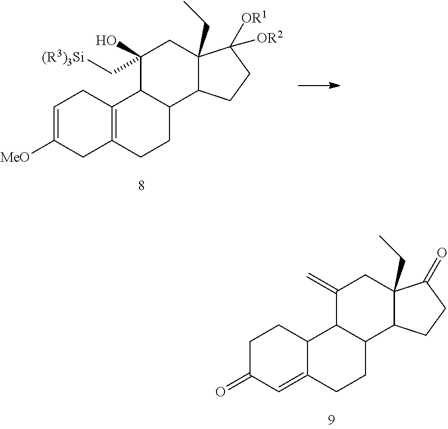

In some embodiments, a compound has the structure 10

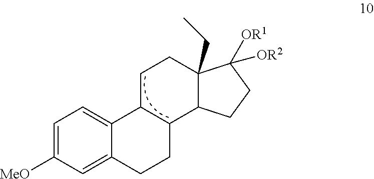

where R¹ and R² are independently C₁-C₆ straight chain or branched alkyl or R¹ and R² together combine to form a cyclic ketal protecting group forming a 5 or 6 members cyclic group comprising an unbranched or branched hydrocarbon.

In some embodiments, a compound has the structure 11

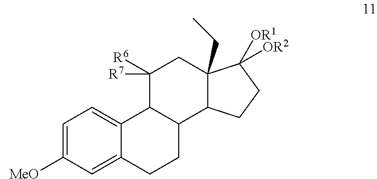

where R⁶ is —OH;
where R⁷ is H or —CH₂—Si(R₃)₃; or
where R⁶ and R⁷ together form ═O;
where R¹ and R² are independently C₁-C₆ straight chain or branched alkyl or R¹ and R² together combine to form a cyclic ketal protecting group forming a 5 or 6 members cyclic group comprising an unbranched or branched hydrocarbon; and
where R³ is an alkyl group.

In some embodiments, a compound has the structure 8

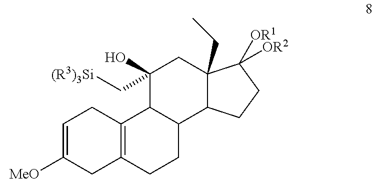

where R¹ and R² are independently C₁-C₆ straight chain or branched alkyl or R¹ and R² together combine to form a cyclic ketal protecting group forming a 5 or 6 members cyclic group comprising an unbranched or branched hydrocarbon; and
where R³ is an alkyl group.

### DETAILED DESCRIPTION OF THE PREFERRED EMBODIMENTS

The commercially available delta 8 estradiol methyl ether 1 was oxidized under Oppenauer conditions with Benzaldehyde, BHT and aluminum isopropoxide to the corresponding 17 ketone 2. The next step acid catalyzed double bond isomerisation afforded a mixture of the delta 9, 11 and delta 8 derivative 3. Protection of the 17-keto group of 3 was performed by converting the 17-keto group into ketal derivative 4, where R¹ and R² are independently C₁-C₆ straight chain or branched alkyl or R¹ and R² together combine to form a cyclic ketal protecting group forming a 5 or 6 members cyclic group comprising an unbranched or branched hydrocarbon. In one embodiment, a 2,2-dimethyl-propane ketal protecting group (R¹ and R² combine to form —CH₂—C(CH₃)₂—CH₂— may be used. The protected intermediate 4 was subjected to selective hydroboration/hydrogen peroxide oxidation to give the 11-hydroxy derivative 5.

Oxidation of the 11-hydroxy compound 5 to 11-keto derivative 6 was accomplished by using either pyridinium chloro chromate on alumina or Swern Oxidation conditions employing dimethyl sulfoxide, trifluoroacetic anhydride and triethylamine at low temperatures. Alternatively, compound 6 may be prepared in high yields by using the Dess-Martin periodinane as the oxidizing agent.

A variety of methods are available in the literature for the conversion of 11-keto derivative 6 to the corresponding 11-methylene derivative 9. These include Wittig reaction, methyl lithium or methyl Grignard addition followed by subsequent water elimination and Peterson elimination reactions. All these methods have their own drawbacks; Wittig reaction using triphenyl phosphonium halide requires sonication conditions as well as the use of large excess of the reagents which may be difficult to supply in large scale synthesis (Tetrahedron, 1994, 50, 10709). Methyl lithium or methyl Grignard addition following the elimination reaction may also produce isomeric olefinic mixtures which are hard to separate. Addition of trimethylsilylmethyl lithium followed by Peterson Olefination appears to be relatively successful for the conversion of a keto group to the corresponding methylene derivative.

I. Synthetic Route for Etonogestrel:

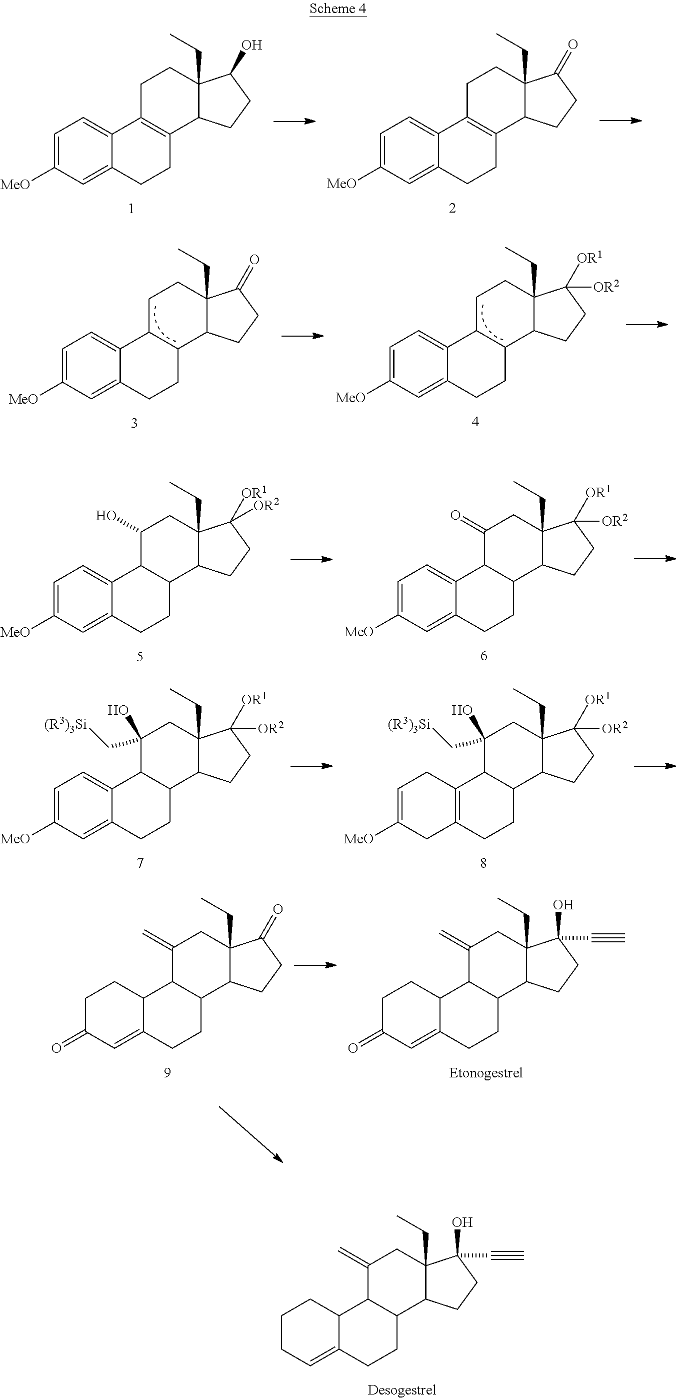

All reported procedures for the addition of trimethylsilylmethyl lithium in an Etonogestrel or Desogestrel synthesis employ very low temperature which in turn is problematic considering large scale synthesis. We have, however found that this reaction can be carried out smoothly at 0° C. in anhydrous toluene affording excellent yields of the 11-silyl-11-alcohol derivative 7, where R³ is an alkyl group. In one embodiment, R³ is methyl. A Birch reduction of compound 7 gave almost quantitative yields of compound 8 which on acid hydrolysis gave 11-methylene diketone 9. Compound 9 may be subsequently transformed to either Desogestrel or Etonogestrel by already known methods (Scheme 4).

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### 13β-Ethyl-3-methoxygona-1,3,5(10),8-tetraen-17-one (2)

A solution of 1 (100 g, 335.1 mmol) in MTBE (750 ml) was stirred under nitrogen as benzaldehyde (59.6 ml, 586.4 mmol) and BHT (1 g, 4.5 mmol) was added. This was followed by the addition of aluminum isopropoxide (145.8 g, 713.7 mmol) and the reaction mixture was stirred at room temperature for 4 h. TLC showed complete conversion of starting material to the product (ΔRf=2 cm, 20% EtOAc-Hexane). The reaction mixture was cooled to 0° C. in an ice bath and carefully quenched with 4M HCl (′500 ml) and was stirred until all the aluminum salts went in to the solution. The layers were separated and the aqueous layer was extracted with MTBE (300 ml×2). The combined organic layers were washed with water (300 ml×2), brine (300 ml) and dried over anhydrous sodium sulfate. The solvent was removed in vacuo to get a crude solid which was suspended in 20% aqueous methanol (1 L). The suspension was mechanically stirred until homogeneous slurry was obtained. This was stored in the refrigerator overnight and filtered. The solid was washed with ice cold 20% aqueous Methanol and dried in vacuum oven overnight to give 82.1g of ketone 2 in 82% yields.

FTIR (ATR): 2918, 2834, 1729, 1607, 1495, 1270, 810 cm⁻¹

¹HNMR (300 MHz, CDCl₃): δ 0.84 (t, J=7.3 Hz, —CH₂—CH₃), 3.81 (s, —OCH₃), 6.72 (m, 2H), 7.13 (d, J=8 Hz, 1H) ppm.

### EXAMPLE 2

### Preparation of mixtures of 13β-Ethyl-3-methoxygona-1,3,5(10),8-tetraen-17-one (2) and (8a)-13β-Ethyl-3-methoxygona-1,3,5(10),9,(11)-tetraen-17-one (3)

A solution of the ketone 2 (82.1 g, 276.9 mmol) in a 3:1 solvent mixture of MeOH and THF (820 ml) was carefully mixed together with con. HCl (410 ml) under nitrogen and was set to reflux for 18h. The reaction was cooled to 0° C. in an ice bath and was carefully neutralized by the addition of concentrated ammonia solution (′342.6 ml). This led to the precipitation of isomeric ketone 3. It was filtered, washed with water and dried in vacuum oven to afford 80.1 g of compound 3 in 97.6% yields. The ratio of Δ-8(9) and Δ-9(11) isomers were found to be 1:2.4 by ¹H NMR.

FTIR (ATR): 2918, 2834, 1731, 1624, 1495, 1237, 812 cm⁻¹

¹HNMR (300 MHz, CDCl₃): (mixture of isomers) δ 0.84 (m, —CH₂—CH₃), 3.79 (s, —OCH₃), 3.81 (s, —OCH₃), 6.11 (m, 1H, 11-H), 6.61 (m, Ar—H), 6.72(m, Ar—H), 7.15 (d, =8.8 Hz, ArH, 1-Hof Δ-8,(9)) 7.52 (d J=8.8 Hz, ArH, 1-Hof Δ-9(11)) ppm.

### EXAMPLE 3

### [(17,17-(2,2-Dimethyl)propane1,3-dioxy]-13β-Ethyl-3-methoxygona-1,3,5(10),8-tetraene and [(17,17-(2,2-Dimethyl)propane1,3-dioxy]-13β-Ethyl-3-methoxygona-1,3,5(10),9,(11)-tetraene (4)

To a solution of the crude mixture of isomeric ketone 3 (80.1 g, 270.2 mmol) in toluene (500 ml) was added neopentyl glycol (154.8 g, 1.5 mol), p-Toluene sulphonic acid (2.6 g, 13.5 mmol) and triethyl orthoformate (157.3 ml, 945.8 mmol). The resulting solution was stirred at room temperature for 48 h. The reaction mixture was carefully quenched with saturated sodium bicarbonate solution and was extracted with ethyl acetate (500 ml). The combined organic layers were washed with water (200 ml), brine (100 ml) and dried over anhydrous sodium sulfate. The solvent was removed under vacuum to afford the crude product as a purple colored solid (103 g, 99%) which was used as such for the next step. A pure sample of this material was obtained by a flash column chromatography on silica column eluting with 10% ethyl acetate hexane solvent system. The ratio of 4-8(9) and Δ-9(11) isomers were found to be 1:2 by ¹H NMR.

FTIR (ATR): 2954, 2871, 1065, 1009, 943 cm⁻¹

¹HNMR (300 MHz, CDCl₃): (mixture of isomers) δ 0.74 (s, —CH₃),0.92 (m, —CH₂—CH₃), 1.03 (m, —CH₂—CH₃), 1.15 (s, —CH₃), 3.39 (m, —OCH₂—), 3.78 (s, —OCH₃), 6.15 (m, 11-H), 6.60 (d, J=2.6 Hz, ArH), 6.71 (dd, J=8.7 Hz, 2.6 Hz, ArH), 7.15,(d, J=8.8 Hz, ArH, −1Hof Δ-8,(9)) 7.52 (d J=8.8 Hz, ArH, 1-Hof Δ-9(11)) ppm.

### EXAMPLE 4

### [(17,17-(2,2-Dimethyl)propane1,3-dioxy]-13β-Ethyl-11α-hydroxy-3-methoxy-gona-1,3,5triene (5)

A solution of compound 4 (49 g, 128.3 mmol) in THF (200 ml) was cooled to 0° C. and was treated drop wise with borane-THF solution (11.02 g, 128.3 mmol). The reaction mixture was stirred for 17 h during which time it was allowed to warm to room temperature. The reaction mixture was cooled to 0° C. and was carefully treated with 10% aqueous NaOH solution (102.6 ml) and 30% hydrogen peroxide solution (29.1 ml). The resulting solution was stirred at room temperature for 4 h after which it was diluted with water (1 L) and extracted with Ethyl Acetate (500 ml×2). The combined organic layers were washed with water (500 ml×2), brine (500 ml) and dried over anhydrous sodium sulfate. The solvent was removed under vacuum to afford ˜50 g of crude 5 and unreacted delta 8-9 derivative as white foam. This material was purified by a flash column chromatography on silica gel eluting with 5% ethyl acetate-hexane solvent mixture to afford 22.48 g (66%) of compound 5 and 32 g of unreacted delta 8-9 compound 4.

FTIR (ATR): 3449, 2943, 2869, 1496, 1125, 1064 cm⁻¹

¹HNMR (300 MHz, CDCl₃): δ 0.73 (s, —CH₃),1.02 (t, J=7.2 Hz —CH₂—CH₃), 1.15 (s, —CH₃), 2.81 (t, J=6.6 Hz) 3.39 (m, —OCH₂—),3.63(d, J=11 Hz) 3.78 (s, —OCH₃), 4.05 (m, 11-H) 6.57 (d, 2.3 Hz, ArH), 6.73 (dd, J=8.9 Hz, 2.9 Hz, ArH), 7.88 (d J=8.6 Hz, ArH, 1-H) ppm.

### EXAMPLE 5

### [(17,17-(2,2-Dimethyl)propane1,3-dioxyl-13β-Ethyl-11-oxo-3-methoxy-gona-1,3,5triene (6)

To a solution of pyridinium chlorochromate (8.1 g, 37.4 mmol) in dry Dichloromethane (25 ml) was added neutral alumina (8.1 g). The resulting slurry was stirred at room temperature for 30 min, after which a solution of 11-alcohol 5 (5 g, 12.5 mmol) in dichloromethane (25 ml) was added drop wise and stirred at room temperature for 4 h. TLC (20% EtOAc-Hexane) showed complete conversion of starting material to the product (ΔRf=1 cm). The reaction mixture was filtered through a short pad of Celite® and Silica and washed with ether (200 ml). The solvent was removed under vacuum to afford a viscous liquid which on trituration with 20% aqueous methanol gave the 11-keto compound as white solid which was filtered and dried (3.23 g, 66.4%)

FTIR (ATR): 3449, 2943, 2869, 1496, 1125, 1064 cm⁻¹

¹HNMR (300 MHz, CDCl₃): δ 0.73 (s, —CH₃),1.02 (t, J=7.2 Hz —CH₂—CH₃), 1.15 (s, —CH₃), 2.81 (t, J=6.6 Hz) 3.39 (m, —OCH₂—),3.63(d, J=11 Hz) 3.78 (s, —OCH₃), 4.05 (m, 11-H) 6.57 (d, J=2.3 Hz, ArH), 6.73 (dd, J=8.9 Hz, 2.9 Hz, ArH), 7.88 (d J=8.6 Hz, ArH, 1-H) ppm.

### EXAMPLE 6

### [(17,17-(2,2-Dimethyl)propane1,3-dioxyl]-13β-Ethyl-11-oxo-3-methoxygona-1,3,5triene (6)

A solution of compound 5 (21.5 g, 53.6 mmol) in THF (215 ml) was treated with DMSO (23 g, 295.5 mmol) and stirred at room temperature for 30 min. The reaction mixture was cooled to −30° C. and trifluoroacetic anhydride (22.6 g, 107.4 mmol) was added drop wise. After stirring for 1 h at −30° C., the reaction was quenched by the addition of triethylamine (24 g, 241.6 mmol). The reaction mixture was warmed to 0° C. and stirred for another 2 h. The reaction mixture was quenched by the addition of water and extracted with ethyl acetate (200 ml×2). The combined organic layer was washed with water (300 ml×1), brine (300 ml×1) and was dried over anhydrous sodium sulfate. The solvent was removed under vacuum to give a viscous liquid which was triturated with ice cold methanol to precipitate the ketone 6 out. The white solid thus obtained was filtered, washed with water and dried to give 12.6 g of pure ketone 6 in 60% yields.

FTIR (ATR): 3449, 2943, 2869, 1496, 1125, 1064 cm⁻¹

¹HNMR (300 MHz, CDCl₃): δ 0.73 (s, —CH₃),1.02 (t, J=7.2 Hz —CH₂—CH₃), 1.15 (s, —CH₃), 2.81 (t, J=6.6 Hz) 3.39 (m, —OCH₂—),3.63(d, J=11 Hz) 3.78 (s, —OCH₃), 4.05 (m, 11-H) 6.57 (d, J=2.3 Hz, ArH), 6.73 (dd, J=8.9 Hz, 2.9 Hz, ArH), 7.88 (d J=8.6 Hz, ArH, 1-H) ppm.

### EXAMPLE 7

### [(17,17-(2,2-Dimethyl)propane1,3-dioxyl]-13β-Ethyl-11β-hydroxy-3-methoxy-11-α-trimethylsilyl-methylgona1,3,5(10)-triene (7)

A solution of compound 7 (16.6 g, 41.6 mmol) in dry toluene (100 ml) at 0° C. was treated drop wise with a 1M solution of trimethylsilylmethyl lithium in pentane and the reaction mixture was stirred for 4 h warming to room temperature. TLC showed complete conversion of starting material. The reaction mixture was cooled to 0° C. and was carefully quenched by the addition of sat. ammonium chloride (50 ml). The organic layer was separated and the aqueous layer was extracted with ethyl acetate (100 ml×2). The combined organic layers were washed with water (100 ml), brine (100 ml) and were dried over anhydrous sodium sulfate sodium sulfate. The solvent was removed under vacuum to afford crude 9 as white foam (18 g). This material was pure enough to take to the next step reaction. A pure sample was obtained by purification though flash column chromatography on silica using 10% EtOAc-Hexane solvent system.

FTIR (ATR): 3584, 2942, 2862, 1246, 1099, 831 cm⁻¹

¹HNMR (300 MHz, CDCl₃): δ 0.12 (s, 9H, -11-CH₂Si(CH₃)₃, 0.73 (s,3H, —CH₃),1.10-14 (m, 6H, —CH₂—CH₃, —CH₃), 1.27 (d, J=15 Hz,1H, 11-CH₂Si—), 1.97(d, J=14.7 Hz,1H, 11-CH₂Si—), 3.29-3.38 (m, 4H), 3.60 (d, J=11 Hz), 3.78 (s, —OCH₃), 6.70-6.72 (m, 2H, ArH), 7.85 (d J=8.7 Hz, ArH, 1-H) ppm.

### EXAMPLE 8

### [(17,17-(2,2-Dimethyl)propane1,3-dioxyl]-13β-Ethyl-11β-hydroxy-3-methoxy-11-α-trimethylsilyl-methylgona1,3-diene (8)

Lithium wire (278 mg, 40 mmol) cut in to small pieces was added to condensed liquid ammonia (40 ml) kept at −40° C. . The resulting deep blue mixture was stirred for 30 minutes. A solution of compound 7 (3.9 g, 8.0 mmol) in THF (43 ml) and ^{t}-BuOH (3.8 ml) was added drop wise so that the deep blue color of the reaction was maintained. The reaction mixture was stirred at −40° C. for 2 h and was carefully quenched by the addition of methanol (′10 ml). Excess ammonia was distilled of at room temperature and the reaction mixture was diluted by the addition of saturated ammonium chloride. The organic layer was separated and the aqueous layer was extracted with ethyl acetate (50 ml×2). The combined organic layers were washed with water, brine and dried over anhydrous sodium sulfate. The solvent was removed under vacuum to afford 8 as white foam (3.85 g, 98%) which was used as such for the next step.

FTIR (ATR): 3584, 2947, 2873, 1247, 1222, 1109, 837 cm⁻¹

¹HNMR (300 MHz, CDCl₃): δ 0.12 (s, 9H, -11-CH₂Si(CH₃)₃, 0.71 (s, 3H, —CH₃),1.07 (t, J=7.5 Hz, —CH₂—CH₃), 1.12 (s, 3H, —CH₃), 2.58-2.62(m, 2H) 3.29-3.38 (m, 4H) 3.54 (s, —OCH₃), 3.59 (d, J=11 Hz), 4.65 (t, J=3.7 Hz, 1H, H-2) ppm.

### EXAMPLE 9

### 13β-Ethyl-11-methylenegon-4en-3,17-dione(9)

A solution of compound 8 (3.85 g, 7.8 mmol) in methanol (35 ml) was cooled to 0° C. and was treated drop wise with con. HCl (1.72 g, 47.2 mmol). The resulting solution was stirred at reflux for 3 h. TLC showed complete conversion of starting material to the product (1:1 EtOAc-Hexane, ΔRf=3 cm). The reaction mixture was cooled to 0° C. and was carefully quenched by the addition of saturated sodium bicarbonate solution. The reaction mixture was extracted with ethyl acetate (50 ml×3). The combined organic layers were washed with water (100 ml×2), brine (100 ml) and were dried over anhydrous sodium sulfate. The solvent was removed under vacuum to give crude 9 as tan foam which was purified by flash chromatography on silica gel using 1:1 EtOAc-Hexane solvent system to afford pure 9 as a white crystalline solid (1.52 g, 65%). Alternately, pure product can also be crystallized from acetone-hexane solvent system.

FTIR (ATR): 2929, 2871, 1725, 1666, 1616, 1213, 915 cm⁻¹

¹HNMR (300 MHz, CDCl₃): δ 0.78 (t, J=7.3 Hz, 3H, —CH₃), 4.90 (s, 1H, 11-CH), 5.02 (s, 1H, 11-CH), 5.9 (s, 1H, 4-H) ppm.

¹³C NMR (75 MHz, CDCl₃): 7.35, 18.08, 20.74, 28.22, 29.40, 35.04, 35.95, 36.86, 37.49, 40.84, 51.89, 52.68, 54.05, 110.37, 125.82, 144.84, 165.69, 199.74, 217.95 ppm.

Mp: 152-154° C.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method for the synthesis of etonogestrel and desogestrel comprising:
converting compound 1 to compound 6; 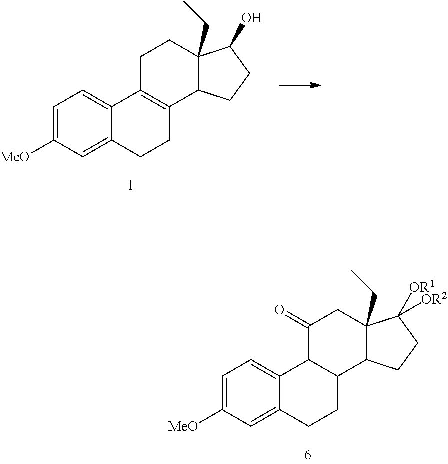
Where
converting compound 6 to compound 9; 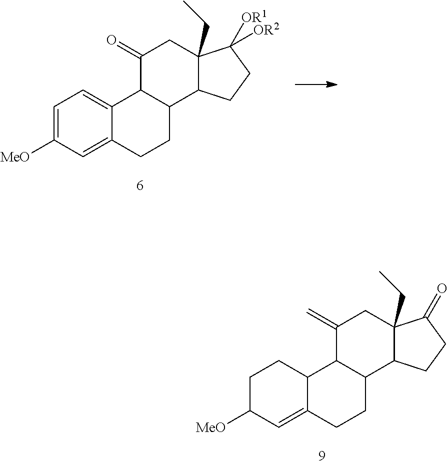
where R¹ and R² are independently C₁-C₆ straight chain or branched alkyl or R¹ and R² together combine to form a cyclic ketal protecting group forming a 5 or 6 members cyclic group comprising an unbranched or branched hydrocarbon; and
converting compound 9 into etonogestrel or desogestrel.

2. The method of claim 1, wherein converting compound 1 to compound 6 comprises:
obtaining or synthesizing compound 1;
oxidizing compound 1 to form compound 2; 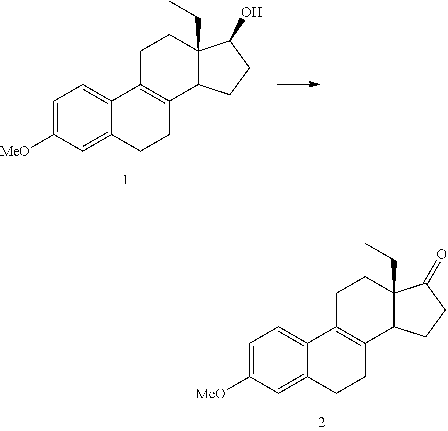 [SMILES: CC[C@]12CCC3=C(CCc4cc(OC)ccc43)C1CC[C@@H]2O ; CC[C@]12CCC3=C(CCc4cc(OC)ccc43)C1CCC2=O]
treating compound 2 with an acid to form the isomerized intermediate 3; and 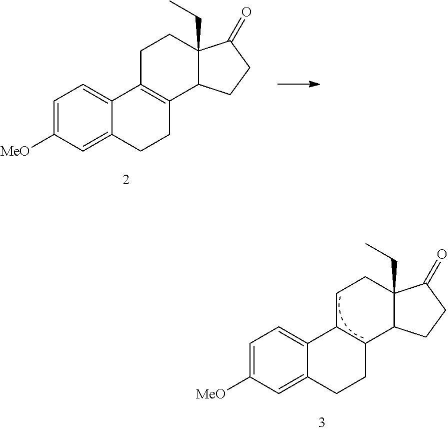 [SMILES: CC[C@]12CCC3=C(CCc4cc(OC)ccc43)C1CCC2=O ; CC[C@]12CCC3c4ccc(OC)cc4CCC3C1CCC2=O]
forming the ketal protected intermediate 4 from compound 3

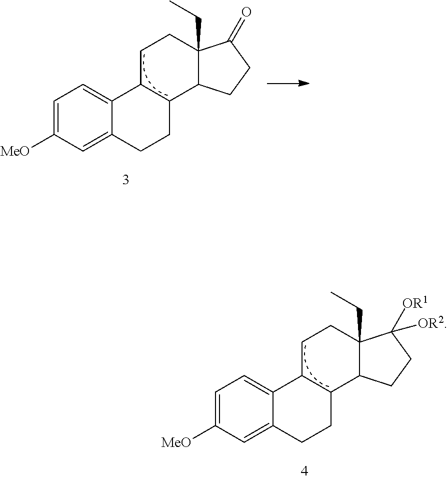

3. The method of claim 2, wherein converting compound 1 to compound 6 further comprises hydroxylating compound 4 to form hydroxy intermediate 5

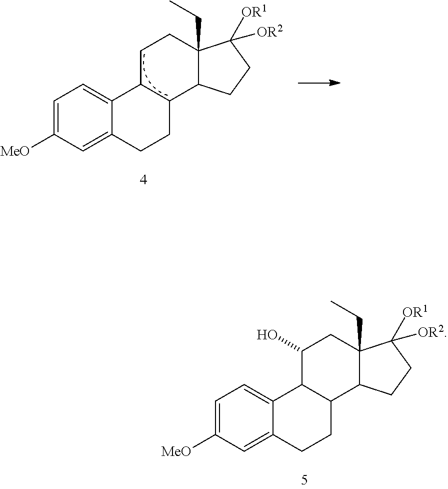

4. The method of claim 3, wherein converting compound 1 to compound 6 further comprises oxidizing the hydroxyl intermediate 5 to form compound 6

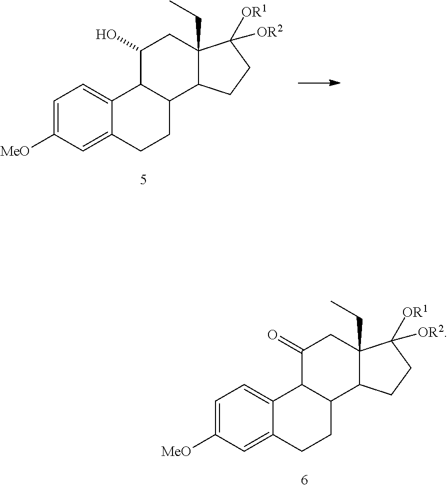

5. The method of claim 1, wherein converting compound 6 to compound 9 comprises forming silyl intermediate 7 from compound 6 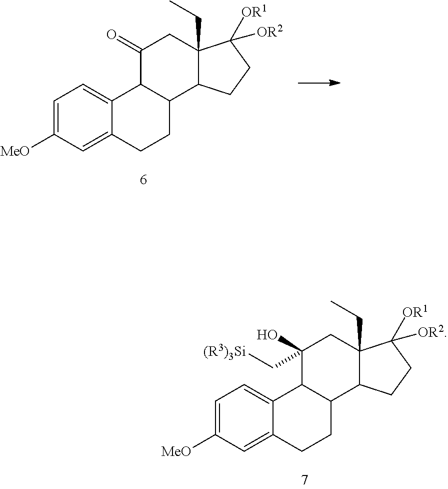
where R³ is an alkyl group.

6. The method of claim 5, wherein converting compound 6 to compound 9 further comprises reducing silyl intermediate 7 to form compound 8

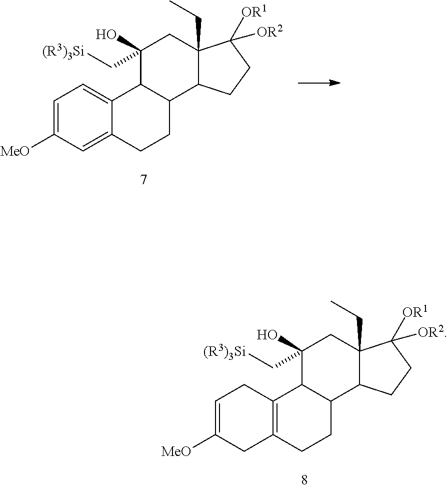

7. The method of claim 6, wherein converting compound 6 to compound 9 further comprises treating compound 8 with acid to form compound 9

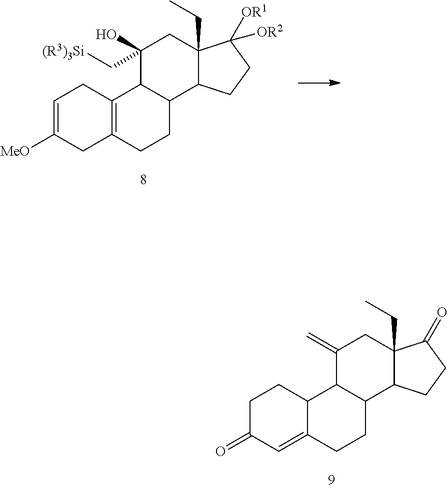

8. The method of claim 1, comprising forming etonogestrel from compound 9.

9. The method of claim 1, comprising forming desogestrel from compound 9.

10. A compound having the structure 10 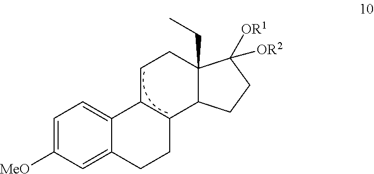
where R¹ and R² are independently C₁-C₆ straight chain or branched alkyl or R¹ and R² together combine to form a cyclic ketal protecting group forming a 5 or 6 members cyclic group comprising an unbranched or branched hydrocarbon.

11. A compound having the structure 11 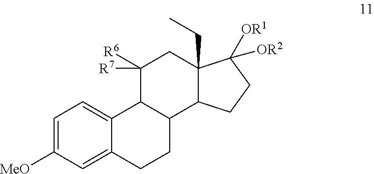
where R⁶ is —OH;
where R⁷ is H or —CH₂—Si(R₃)₃; or
where R⁶ and R⁷ together form ═O;
where R¹ and R² are independently C₁-C₆ straight chain or branched alkyl or R¹ and R² together combine to form a cyclic ketal protecting group forming a 5 or 6 members cyclic group comprising an unbranched or branched hydrocarbon; and
where R³ is an alkyl group.

12. A compound having the structure 8 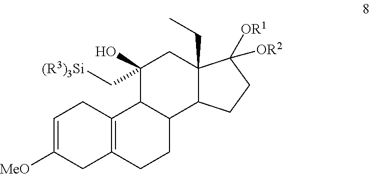
where R¹ and R² are independently C₁-C₆ straight chain or branched alkyl or R¹ and R² together combine to form a cyclic ketal protecting group forming a 5 or 6 members cyclic group comprising an unbranched or branched hydrocarbon; and
where R³ is an alkyl group.
